(19) United States
(12) Patent Application Publication
(10) Pub. No.: US 2025/0121171 A1
(43) Pub. Date: Apr. 17, 2025

(54) PORT LOCATOR FOR AN IMPLANT

(71) Applicant: Mentor Worldwide LLC, Irvine, CA (US)

(72) Inventors: Michael HOFFMAN, Hillsborough, NJ (US); Annmarie MULLEN, Bridgewater, NJ (US); Lucas HELDMAN, Wayne, NJ (US); Josvan RODRIGUEZ, Laguna Hills, CA (US); Shengkai PENG, Trophy Club, TX (US); Omar RODRIGUEZ, Lantana, TX (US)

(73) Assignee: Mentor Worldwide LLC, Irvine, CA (US)

(21) Appl. No.: 18/378,852

(22) Filed: Oct. 11, 2023

(51) Int. Cl. A61M 39/02; A61B 90/00

(52) U.S. Cl. CPC A61M 39/0208; A61B 90/02; A61M 2039/0238; A61M 2205/0272

(57) ABSTRACT

An implant port locator tool for locating an injection port of an implant within a patient includes a housing having a base. A second side of the base has a recess. A cage has an outer perimeter connected to the second side of the base. The cage has a recess and a central opening in the recess. The recess in the base and the recess in the cage mate to form a receptacle. A pin has a first end and a second end. The first end of the pin is disposed within the receptacle. The second end of the pin is disposed exterior to the recess via the central opening in the cage. A magnet is connected to the first end of the pin. The magnet and the first end of the pin are captured in the receptacle.

### FIELD

The present invention generally relates to an apparatus and method for an implant port locator tool for locating an injection port of an implant within a patient, and more particularly, to a method and apparatus for guiding an external needle to an injection port of an implantable medical device, such as, for example, a breast implant.

### BACKGROUND

This section provides background information related to the present disclosure which is not necessarily prior art.

Implantable or implanted medical devices are often placed sub-dermally in a body of a patient. The implanted medical device can be provided for various purposes but include implanted breast implants, including tissue expanders, and infusion devices that may, for example, include an implantable pump. A tissue expander may periodically require additional fluid, such as, for example, saline to expand the volume of the implant slowly over time. Tissue expanders are typically formed of a silicone polymer shell. After implantation, a fluid, such as saline, is periodically injected into the tissue expander to enlarge it over time. Between injections, the surrounding skin is permitted to stretch and grow to create the increased skin surface and the increased tissue pocket for receipt of a permanent implant. Typically, a tissue expander has an injection element through which fluid can be introduced into or withdrawn from the shell of the tissue expander. One such injection element is an integrated port having a septum that can be pierced with a hypodermic needle for adding or withdrawing fluid from the tissue expander. Alternatively, the injection element may be a self-sealing area on the tissue expander which allows penetration by a hypodermic needle and self-closing after the needle has been withdrawn from the expander.

Implanted pumps infuse a material, such as a functional material or functional fluid into the patient at a predetermined location within the body. The pump can be programmed to infuse the functional material at a selected rate, based on a selected physiological sensing, etc. Over a period of time, however, the reservoir in the implanted medical device may empty thus requiring the reservoir to be refilled to continue operation.

Adding fluid to a tissue expander or refilling an implanted medical device requires accessing the reservoir of the implanted medical device. Accessing the reservoir, however, may be difficult and require open access to the implanted device. Selected devices include ports through which a needle can be placed. The port can sometimes be found through the dermis of the patient by palpation. However, this method can lack the ability to determine the orientation of the external filling or refilling system relative to the implanted medical device and the precise location of the implant port.

### SUMMARY

This section provides a general summary of the present disclosure and is not a comprehensive disclosure of its full scope or all its features.

A port locator in accordance with the present invention can determine a precise orientation and position of where to place and then insert a needle into the skin of a patient to deliver a fluid to an implanted or implantable medical device (IMD). The IMD can include one or more magnets that transmit a magnetic field. The external port locator can include a magnet that, when aligned with the magnet in the implant, can indicate to the user the location on the skin to penetrate with a needle to access the inlet port of the implant.

In some embodiments, an implant port locator tool for locating an injection port of an implant within a patient is disclosed. The implant locator tool comprises a housing having a base. The base has a first side and a second side. The first side of the base is configured to be placed on the skin of the patient. The second side of the base has a recess. A handle is connected to the second side of the base. A cage has an outer perimeter connected to the second side of the base. The cage has a recess and a central opening in the recess. The recess in the base and the recess in the cage mate to form a receptacle with the central opening being disposed at an apex of the receptacle, spaced a predetermined distance from the base. A pin has a first end and a second end. The first end of the pin is disposed within the receptacle. The second end of the pin is disposed exterior to the recess via the central opening in the cage. A magnet is connected to the first end of the pin. The magnet and the first end of the pin are captured in the receptacle.

In some embodiments, the implant locator tool recess in the base has a hemispherical shape.

In some embodiments, the implant locator tool recess in the cage has a hemispherical shape.

In some embodiments, the implant locator tool receptacle has a spherical shape.

In some embodiments, the implant locator tool magnet and the first end of the pin are captured in the receptacle in a ball and socket connection.

In some embodiments, the implant locator tool magnet is an annular ring magnet.

In some embodiments, the implant locator tool magnet is press fit onto the first end of the pin.

In some embodiments, the implant locator tool first end of the pin has an enlarged semispherical head at its distal end.

In some embodiments, the implant locator tool magnet is adjacent to the semispherical head at the first end of the pin.

In some embodiments, the implant locator tool second end of the pin has a position indicator connected to the distal second end of the pin, the pin and the position indicator are configured to be aligned approximately perpendicular to the housing base first side when the implant locator tool is aligned with the injection port of the implant.

In some embodiments, the implant locator tool first side of the base has a crosshair shaped projection projecting away from the first side of the base by a predetermined distance.

In some embodiments, the implant locator tool a center of the crosshair shaped projection aligned with an axis of the pin when the implant locator tool is aligned with the injection port of the implant.

In some embodiments, the implant locator tool the base has a through hole disposed between the handle and the cage.

In some embodiments, the implant locator tool an outer perimeter of the base has at least one semicylindrical recess.

In some embodiments, the implant locator tool second end of the pin has a position indicator connected to the distal second end of the pin. The position indicator is configured to be aligned approximately perpendicular to the housing base first side when the implant locator tool is aligned with the injection port of the implant. The first side of the base has a crosshair shaped projection projecting away from the first side of the base by a predetermined distance. A center of the crosshair shaped projection is aligned with an axis of the pin when the implant locator tool is aligned with the injection port of the implant. An imaginary line extending from one of the crosshair projections intersects the through hole in the base. An imaginary line extending from one of the crosshair projections intersects the at least one semicylindrical recess in the outer perimeter in the base.

In some embodiments, an implant port locator tool for locating an injection port of an implant within a patient comprises a first plate having a first side and a second side. The first side of the first plate is configured to be placed on the skin of the patient. The second side of the first plate has a recess. A second plate has a first side and a second side. The first side of the second plate is connected to the second side of the first plate. The first side of the second plate has a recess and a central opening in the recess opening to the second side of the second plate. The recess in the first plate and the recess in the second plate mate to form a receptacle. A pin has a first end and a second end. The first end of the pin is disposed within the receptacle. The second end of the pin is disposed exterior to the recess via the central opening in the second plate. A magnet is connected to the first end of the pin. The magnet and the first end of the pin are captured in the receptacle.

In some embodiments, the implant locator tool recess in the first plate has a hemispherical shape.

In some embodiments, the implant locator tool recess in the second plate has a hemispherical shape.

In some embodiments, the implant locator tool receptacle has a spherical shape.

In some embodiments, the implant locator tool magnet and the first end of the pin are captured in the receptacle in a ball and socket connection.

In some embodiments, the implant locator tool magnet is an annular ring magnet.

In some embodiments, the implant locator tool magnet is press fit onto the first end of the pin.

In some embodiments, the implant locator tool first end of the pin has an enlarged semispherical head at its distal end.

In some embodiments, the implant locator tool magnet is adjacent to the semispherical head at the first end of the pin.

In some embodiments, the implant locator tool second end of the pin has a position indicator connected to the distal second end of the pin. The pin and position indicator are configured to be aligned approximately perpendicular to the first side of the first plate when the implant locator tool is aligned with the injection port of the implant.

In some embodiments, the implant locator tool first side of the first plate has a crosshair shaped projection projecting away from the first side of the first plate by a predetermined distance.

In some embodiments, the implant locator tool has a center of the crosshair shaped projection aligned with an axis of the pin when the implant locator tool is aligned with the injection port of the implant.

In some embodiments, the implant locator tool first plate and the second plate has four through holes spaced symmetrically about the receptacle.

In some embodiments, the implant locator tool first plate and the second plate have four through holes spaced symmetrically about the receptacle, wherein an imaginary line drawn through the centers of two of the through holes intersects with the center of the crosshair shaped projection.

In some embodiments, an imaginary line drawn through the centers of the other two of the through holes intersects with the center of the crosshair shaped projection.

In some embodiments, the implant locator tool has a handle connected to the first plate.

In some embodiments, a method of locating an injection port of an implant within a patient with an implant port locator tool is disclosed. The method comprises a housing having a base. The base has a first side and a second side. The first side of the base is configured to be placed on the skin of the patient. The second side of the base has a recess. A handle is connected to the second side of the base. A cage, having an outer perimeter, is connected to the second side of the base. The cage has a recess and a central opening in the recess. The recess in the base and the recess in the cage mate to form a receptacle with the central opening being disposed at an apex of the receptacle, spaced a predetermined distance from the base. A pin has a first end and a second end. The first end of the pin is disposed within the receptacle. The second end of the pin is disposed exterior to the recess via the central opening in the cage. A magnet is connected to the first end of the pin. The magnet and the second end of the pin are captured in the receptacle. The method comprises the steps of: placing the first side of the base of the implant port locator tool on the skin of a patient having an implant with an injection port; moving the implant port locator tool on the skin of the patient until the pin and the position indicator are aligned approximately perpendicular to the housing base first side to indicate that the implant locator tool is aligned with the injection port of the implant; marking the skin of the patient to indicate the location of the injection port of the implant under the skin.

In some embodiments, the implant locator tool includes the base having a through hole disposed between the handle and the cage. An outer perimeter of the base has at least one semicylindrical recess. Marking the skin of the patient step further comprises using a marker through the through hole and at the at least one semicylindrical recess to mark the patient's skin.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A is a partial cross-section of a tissue expander implanted in a patient and an external port locator being located on the skin of the patient.

FIG. 1B is a partial cross-section of a tissue expander implanted in a patient and an external port locator being located on the skin of the patient.

FIG. 2 is a partial cross-section of a tissue expander implanted in a patient and a syringe and needle for injection fluid into or removing fluid from an injection port of the tissue expander.

FIG. 3 is a perspective view of the port locator tool according to the present invention.

FIG. 4 is a partial cross-sectional view of the port locator tool of FIG. 1.

FIG. 5 is a top view of the port locator tool of FIG. 1.

FIG. 6 is a bottom view of the port locator tool of FIG. 1.

FIG. 7A is a cross-sectional view of the port locator tool of FIG. 1.

FIG. 7B is a cross-sectional view of the port locator tool of FIG. 1.

FIG. 7C is a cross-sectional view of the receptacle of the port locator tool.

FIG. 8 is a perspective view of another embodiment of a port locator tool.

FIG. 9 is a cross-sectional view, taken along line 9-9 of FIG. 8 and looking in the direction of the arrows.

FIG. 10 is a bottom view of the port locator tool of FIG. 8.

FIG. 11 is a perspective view of another embodiment of a port locator tool.

FIG. 12 is a partial cross-sectional view of the port locator tool of FIG. 11.

FIG. 13 is an illustration of an impression made on the skin of a patient from the crosshair shaped projections and marks made on the skin via a through hole and recesses in the port locator tool.

FIG. 14 is a partial perspective view of another embodiment of a port locator tool.

FIG. 15 is a partial perspective view of another embodiment of a port locator tool.

FIG. 16 is a partial perspective view of another embodiment of a port locator tool.

FIG. 17 is a partial perspective view of another embodiment of a port locator tool.

FIG. 18 is a partial perspective view of another embodiment of a port locator tool.

FIG. 19 is a cross-sectional view, taken along line 19-19 of FIG. 18 and looking in the direction of the arrows.

FIG. 20 is a partial perspective view of another embodiment of a port locator tool.

FIG. 21 is a partial perspective view of another embodiment of a port locator tool.

FIG. 22 is a partial perspective view of another embodiment of a port locator tool.

FIG. 23 is a partial perspective view of another embodiment of a port locator tool.

FIG. 24 is a partial perspective view of another embodiment of a port locator tool.

FIG. 25 is a partial perspective view of another embodiment of a port locator tool.

FIG. 26 is a partial perspective view of another embodiment of a port locator tool.

FIG. 27 is a flow chart illustrating a method of using the port locator tool.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±20% of the recited value, e.g., “about 90%” may refer to the range of values from 81% to 99%.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

As used in the specification and the appended claims, the singular forms “a,” “an,” and “the” include plural referents unless the context clearly dictates otherwise. Ranges can be expressed herein as from “about” or “approximately” one particular value and/or to “about” or “approximately” another particular value. When such a range is expressed, other exemplary examples include from the one particular value and/or to the other particular value.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

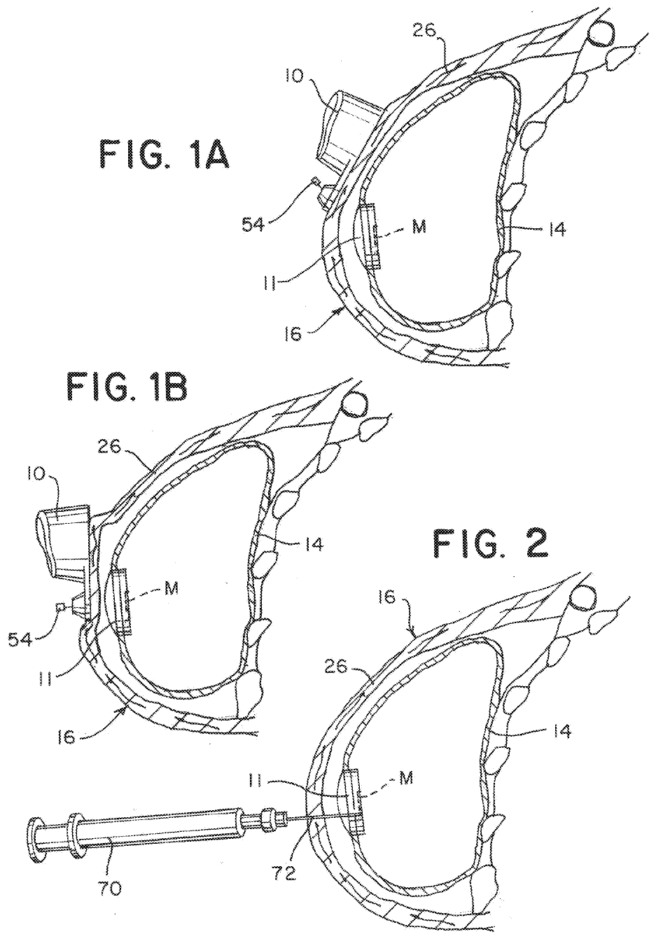

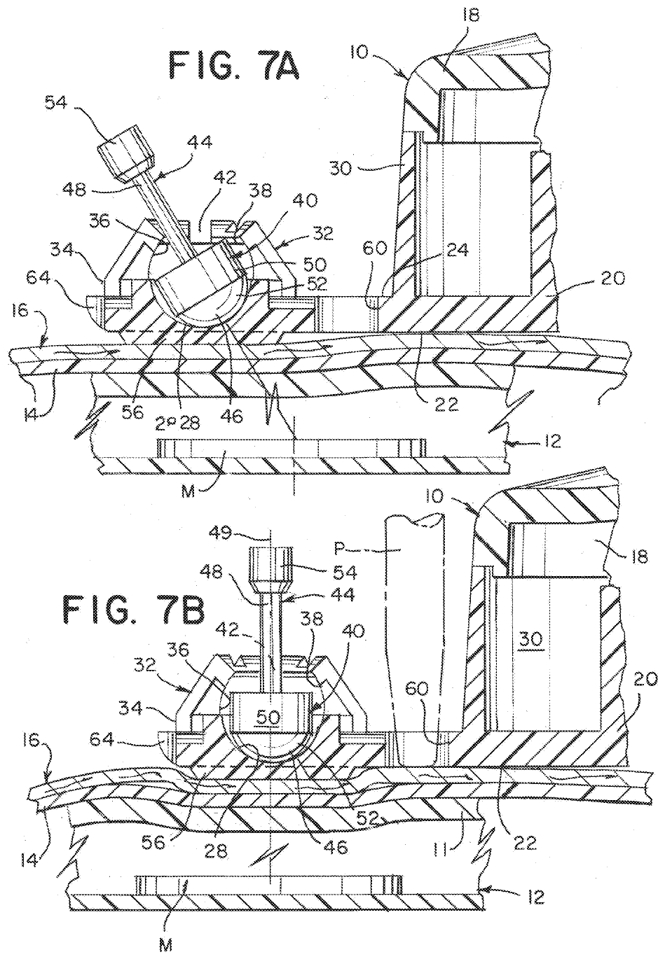

Referring now to FIGS. 1A and 1B, an illustration of a port locator tool 10 for locating an injection port 11 of an implant 14 within a patient 16 in accordance with the present invention is illustrated. Implant 14 may be a tissue expander that may be used in breast reconstruction surgery. Port locator tool is shown on the skin 26 just above the location of injection port 11. In FIG. 1A, port locator tool 10 is shown on the skin 26 of a patient in a location that is not aligned with the injection port 11 of implant 14. In FIG. 1B, port locator tool 10 is shown on the skin 26 of a patient in a location that is aligned with the injection port 11 of implant 14. A position indicator 54, described in greater detail below, is free to move to a limited extent. A magnet M is located within the injection port 11 of implant 14 as shown in FIGS. 1A, 1B, 4, 7A, 7B and 12. When port locator tool is located above the injection port 11 of implant 14, the position indicator 54 is approximately perpendicular to a base of port locator tool 10 as can be seen in FIGS. 1B and 7B. When port locator tool is located on the skin 26, but not above the injection port 11 of implant 14, the position indicator 54 is disposed at an acute angle with respect to a base of port locator tool 10 as can be seen in FIG. 1A. Once the user has located the injection port by using tool 10 as described below, the user may utilize a syringe and needle to inject or remove fluid, such as, for example, saline, to the interior of the implant 14 by penetrating the skin 26 at the location identified by the user in FIG. 1B and then penetrating the implant port 11 as shown in FIG. 2.

Referring now to FIG. 2, an illustration of a syringe and needle for injection fluid into or removing fluid from an injection port 11 of an implant 14 within a patient 16 in accordance with the present invention is illustrated. Implant 14 may be a tissue expander that may be used in breast reconstruction surgery. Syringe 70 is shown on the skin 26 just above the location of injection port 11 with needle 72 penetrating the skin and the injection port 11 to place fluid into or remove fluid from implant 14.

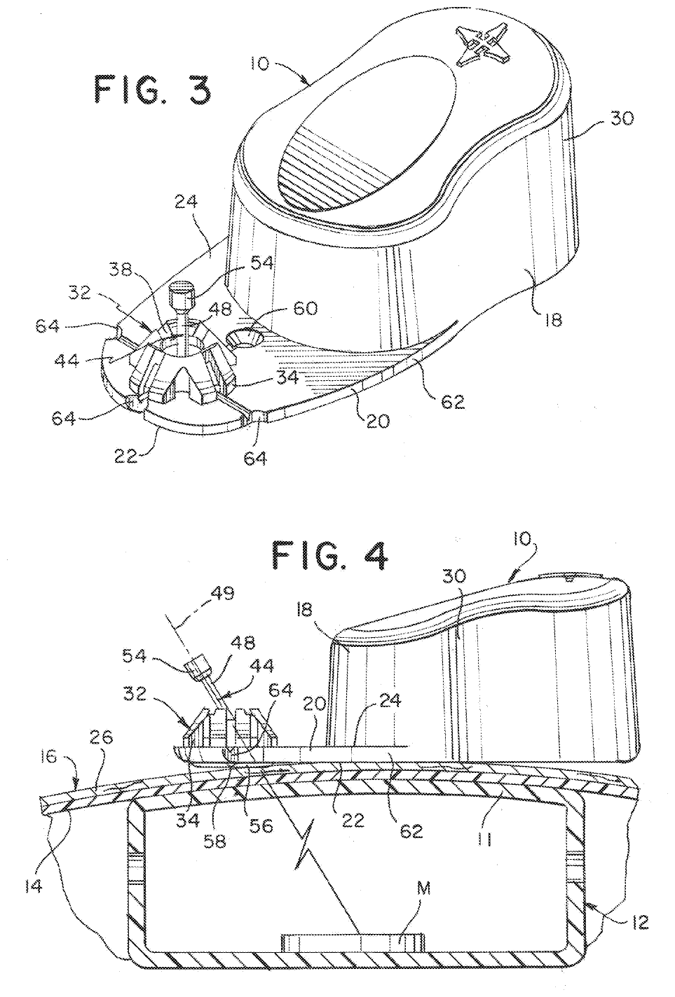

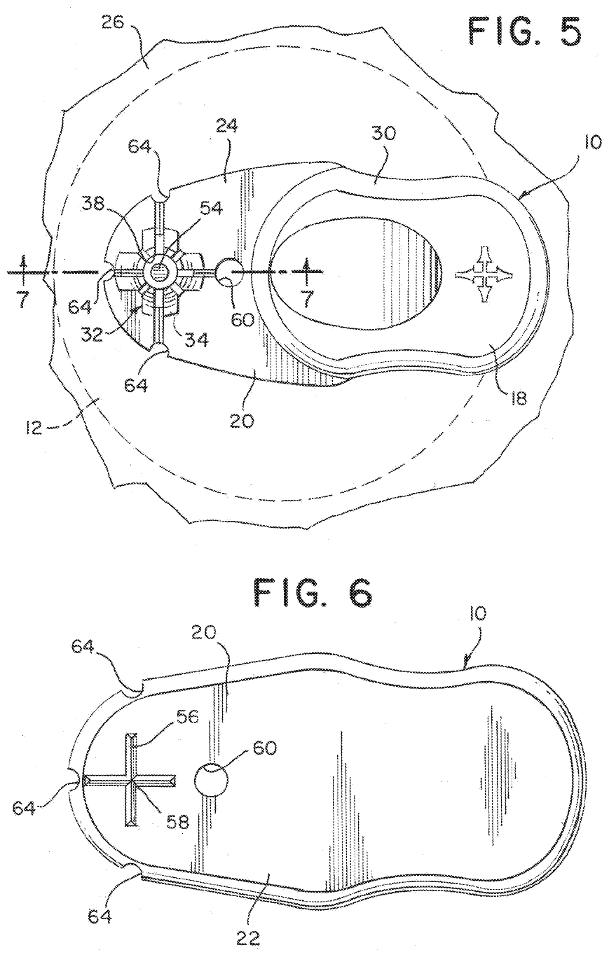

Referring now to FIGS. 3-7C, a port locator tool 10 for locating an injection port 11 of an implant 14 within a patient 16 in accordance with the present invention is illustrated. Implant locator tool 10 comprises a housing having a base 20. Base 20 has a first side 22 and a second side 24. The first side 22 of base 20 is configured to be placed on the skin 26 of patient 16. The second side 24 of base 20 has a recess 28 at one end of the base 20. A handle 30 is also connected to the second side 24 of base 20 at an opposite side of the base 20 from recess 28. As shown in FIGS. 7A-7C, a cage 32 has an outer perimeter 34 connected to the second side 24 of base 20. Cage 32 has a recess 36 and a central opening 38 in recess 36. Recess 28 in base 20 and recess 36 in cage 32 mate to form a receptacle 40 with central opening 38 being disposed at an apex 42 of receptacle 40. Apex 42 is spaced a predetermined distance from base 20. A pin 44 has a first end 46 and a second end 48. The first end 46 of pin 44 is disposed within receptacle 40. The second end 48 of pin 44 is disposed exterior to recess 36 and receptacle 40 via the central opening 38 in cage 32. A magnet 50 is connected to the first end 46 of pin 44. Magnet 50 and the second end 48 of pin 44 are captured in receptacle 40.

Recess 28 in base 20 preferably has a hemispherical shape. The recess 36 in cage 32 also preferably has a hemispherical shape. Recess 28 in base 20 and recess 36 in cage 32 mate to form receptacle 40, which has a generally spherical shape. Magnet 50 and the first end 46 of the pin 44 are captured in receptacle 40 in a ball and socket connection, as shown in FIGS. 7A-7C. Absent any external forces, the first end 46 of pin 44 is free to move within receptacle 40 to the degrees shown in FIGS. 7A and 7B. The movement of pin 44 is limited by the size of opening 38 in recess 36. As shown in FIGS. 7A and 7B, magnet 50 can be in the shape of an annular ring. Magnet 50 is fixedly connected to the first or proximal end 46 of pin 44. Magnet 50 may be press fit onto the first end 46 of the pin 44. Alternatively, magnet 50 may be welded or glued to the first end 46 of pin 44. Magnet 50 can be fixedly connected to pin 44 by any other means known to those skilled in the art. As shown in FIGS. 7A and 7B, the first end 46 of the pin 44 can have an enlarged semispherical head 52 at its distal end. Magnet 50 is adjacent to the semispherical head 52 at the first end 46 of the pin 44. Semispherical head 52 is sized and shaped to mate with recess 28 so that pin 44 can move freely within receptacle 40 within the limits created by central opening 38.

Alternatively, the magnet may be a spherical shaped magnet 51 as shown in FIG. 7C. In this embodiment there is no semispherical head and magnet 51 is fixedly connected to the proximal first end of pin 44.

As shown in FIGS. 3, 5 and 6, base 20 has a through hole 60 disposed between handle 30 and cage 32. An outer perimeter 62 of base 20 has at least one semicylindrical recess 64, and preferably three recesses 64. The second end 48 of pin 44 has a position indicator 54 connected to the distal second end 48 of pin 44. Pin 44 and position indicator 54 are configured to be aligned approximately perpendicular to the first side 22 of housing base 20 when the implant locator tool 10 is aligned with the injection port 11 of implant 14 as shown in FIG. 7C. because magnet 50 or spherical magnet 51 are attracted to magnet M in implant injection port 11. First side 22 of base 20 has a crosshair shaped projection 56 projecting away from first side 22 of base 20 by a predetermined distance. A center 58 of the crosshair shaped projection is aligned with an axis 49 of pin 44 when the implant locator tool 10 is aligned with the injection port 11 of implant 14.

A position indicator 54 is connected to the distal second end 48 of pin 44. Position indicator 54 is configured to be aligned approximately perpendicular to the first side 22 of housing base 20 when the implant locator tool 10 is aligned with the injection port 11 of implant 14. First side 22 of base 20 has a crosshair shaped projection 56 projecting away from the first side 22 of base 20 by a predetermined distance. A center 58 of the crosshair shaped projection 56 is aligned with the axis 49 of pin 44 when the implant locator tool 10 is aligned with the injection port 11 of implant 14. As shown in FIG. 6, an imaginary line extending from one of the crosshair projections 56 intersects the through hole 60 in base 20 and a semicylindrical recess 64. An imaginary line extending from the other one of the crosshair projections 56 intersects the other two semicylindrical recesses 64 in the outer perimeter 62 of base 20. The intersection of these two imaginary lines is the location on the skin to penetrate with a needle to inject or remove fluid to and from the implant when the port locator is aligned with the injection port 11 as shown in FIGS. 1B and 2.

Magnet M in the implant is a disc shaped magnet and has a size of less than or equal to about 0.375 inches in diameter and a height of less than or equal to about 0.1875 inches, preferably 0.25 inches in diameter and 0.125 inches in height, to be an MR conditional tissue expander with an embedded port magnet. Port locator magnet 50, preferably has a diameter of about 0.25 inches or less and a height of about 0.125 inches or less. Port locator spherical magnet 51 preferably has a diameter of about 0.125 inches or less. An implant, such as, for example, a tissue expander, can be labeled to be MR conditional in the United States if it meets the classification requirements in ASTM F2503-20, the disclosure of which is hereby incorporated by reference. Magnet M, and thus the injection port 11 of implant 14, can be found by the handheld port locator 10 of the present invention, which includes a more sensitive magnet-based ball and socket mechanism to locate the injection port 11.

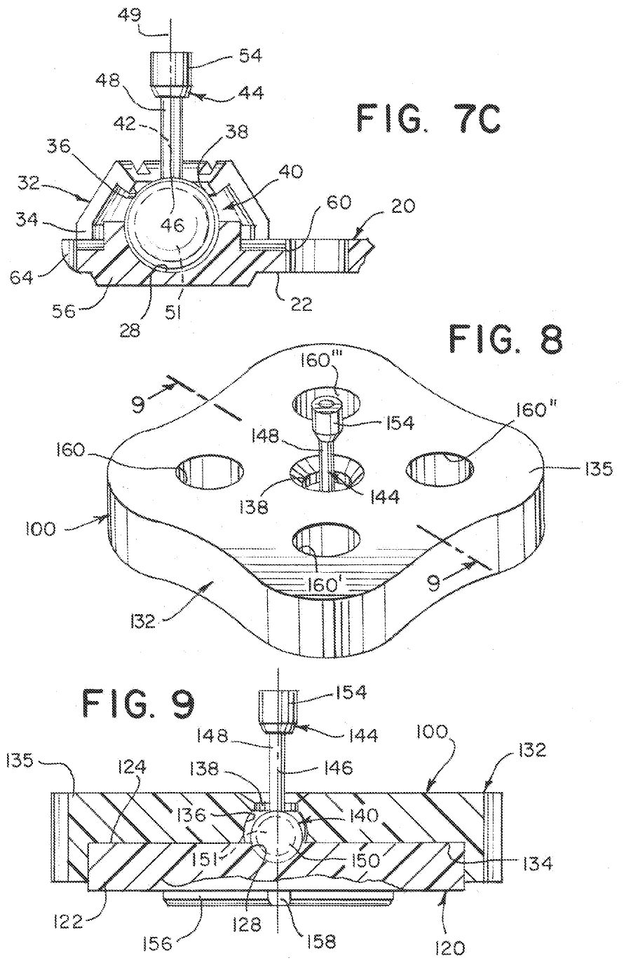

Referring now to FIGS. 8-10, an implant port locator tool 100 is disclosed. Implant locator tool 100 includes a housing having a first plate 120 and a second plate 132. First plate 120 has a first side 122 and a second side 124. The first side 122 of first plate 120 is configured to be placed on the skin 26 of patient 16. Second side 124 of first plate 120 has a recess 128. Second plate 132 has a first side 134 and a second side 135. First side 134 of second plate 132 is connected to the second side 124 of first plate 120. First side 134 of second plate 132 has a recess 136 and a central opening 138 in recess 136. Central opening 138 opens to the second side 135 of second plate 132. Recess 128 in first plate 120 and recess 136 in second plate 132 mate to form a receptacle 140. A pin 144 has a first end 146 and a second end 148. First end 146 of pin 144 is disposed within receptacle 140. Second end 148 of pin 144 is disposed exterior to recess 136 via the central opening 138 in second plate 132. A spherical magnet 151 is connected to the first end 146 of pin 144. Magnet 151 and the first end 146 of pin 144 are captured in receptacle 140.

Recess 128 in first plate 120 preferably has a hemispherical shape. Recess 136 in second plate 132 preferably has a hemispherical shape. Recesses 128, 136 mate to form generally spherically shaped receptacle 140. Magnet 151 and first end 146 of pin 144 are captured in receptacle 140 in a ball and socket connection. Magnet 151 is shown in FIG. 9 as being a spherical magnet to mate with the shape of the generally spherical shaped receptacle 140. Magnet 151 is fixedly connected to first end 146 of pin 144. Magnet 151 is preferably press fit onto the first end 146 of pin 144. Alternatively, the first end 146 of pin 144 can have an enlarged semispherical head at its distal end. An annular ring-shaped magnet can be disposed adjacent to the semispherical head at the first end 146 of pin 144 similar to the embodiment shown and described in FIGS. 7A and 7B.

The second end 148 of pin 144 has a position indicator 154 connected to the distal second end 148 of pin 144. Pin 144 and position indicator 154 are configured to be aligned approximately perpendicular to the first side 122 of first plate 120 when the implant locator tool 100 is aligned with the injection port 11 of implant 14 as shown, for example, in FIGS. 8 and 9. First side 122 of first plate 120 has a crosshair shaped projection 156 projecting away from the first side of first plate 120 by a predetermined distance. A center 158 of the crosshair shaped projection 156 is aligned with an axis of pin 144 when the implant locator tool 100 is aligned with the injection port 11 of implant 14. First plate 120 and second plate 132 each have four through holes 160, 160′, 160″, 160′″ spaced symmetrically about the receptacle 140. An imaginary line drawn through the centers of two of the through holes 160, 160″ intersects with the center 158 of the crosshair shaped projection 156. An imaginary line drawn through the centers of the other two of the through holes 160′, 160′″ intersects with the center 158 of the crosshair shaped projection 156.

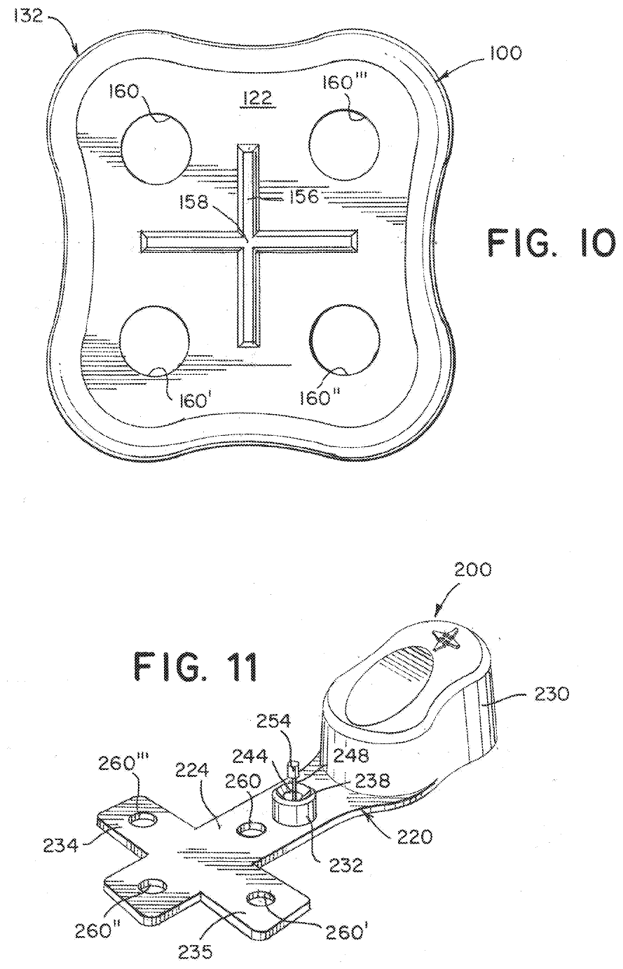

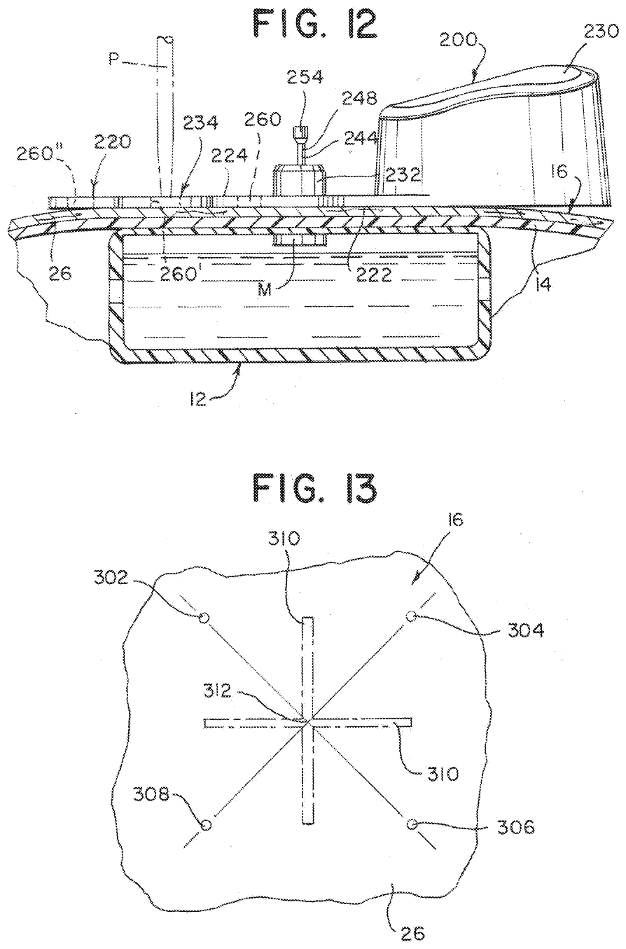

Referring now to FIGS. 11 and 12, an alternative embodiment of an implant port locator tool 200 is illustrated. Tool 200 includes a housing having a first plate 220, a second plate 234 and a third plate 235. Second plate 234 and third plate 235 are fixedly connected to first plate 220. A handle 230 may be connected to first plate 220 as shown in FIGS. 11 and 12. First plate 220 has a first side 222 and a second side 224. The first side 222 of first plate 220 is configured to be placed on the skin 26 of patient 16. Second side 224 of first plate 220 has a cage 232 that is similar to cage 32 described above and illustrated in FIGS. 3-7C and no further description of the ball and socket connection of a pin 244 and a magnet M is included as the structure is similar to cage 32 but being a one-piece cage design. A central opening 238 in cage 232 opens to the second side 224 of first plate 220. A pin 244 has a first end 246 (not shown in FIGS. 11 and 12 but is similar to first end 46 on FIGS. 3-7C) and a second end 248. First end 246 of pin 244 is disposed within a receptacle (not shown) of cage 232. Second end 248 of pin 244 is disposed exterior to cage 232 via the central opening 238. A position indicator 254 is connected to the second end 248 of pin 244. A magnet, not shown, is connected to the first end 246 of pin 244. The magnet and the first end 246 of pin 244 are captured in receptacle 240 in a ball and socket type connection just like in the embodiments shown and described in FIGS. 3-7C and 8-10 and are not described further herein for the sake of brevity in the disclosure.

First plate 220 has two through holes 260, 260″. Second plate 234 has one through hole 260′″. Third plate 235 has one through hole 260′. In this embodiment, a magnet can be relatively smaller and, thus, weaker, which will improve MR conditional outcomes. Magnet M is located more superficially in the port, closer to the skin of the patient, from its typical position in the base of the inlet port 11. In this embodiment, the magnet is centrally located within the port 11. To avoid the needle puncturing and hitting the magnet M, the center of the offset holes 260, 260′, 260″ and 260′″ of the locator tool 200 aligns with a region of open port space between the magnet M and the outer perimeter of port 11. Because the magnet M is centrally located, if the port locator cage is centered over the magnet, the port locator can be rotated at any angle 360 degrees around the port center where the magnet M is located and the holes 260, 260′, 260″ and 260′″ would still demarcate an appropriate position between the magnet M and the side wall of the port 11.

Referring now to FIG. 13, an impression made on the skin 26 of patient 16 from the crosshair shaped projections 56, 156 and marks made on the skin via through hole 60 and recesses 64 or from through holes 160, 160′, 160″, 160′″. Similar impressions and markings on the skin would be made by the embodiment of FIGS. 8-10 except the crosshair impression of FIGS. 8-10 are offset by 45 degrees from the impressions made from the embodiment of FIGS. 3-7C. The illustration of FIG. 10 shows the impression and marks made from the embodiment of FIGS. 8-10. A marker or pen P may be used, by the surgeon or on the surgeon's behalf, to mark the skin at locations 302, 304, 306 and 308. Crosshair shaped projections 56, 156 may leave a temporary mark or impression on the skin 26 as illustrated by impressions 310. An imaginary line, or a real line, if so marked by the surgeon or on the surgeon's behalf, from marks 302, 306 and from marks 304, 308 intersect at the center 312 of the crosshair impressions 310 to provide a second source, or confirmation on where on the skin to penetrate with a needle to access the inlet port 11 of implant 14. The embodiment of FIGS. 3-7C would have similar impressions and marks as shown in FIG. 10, except that the crosshair impression would align with the marks 302, 304, 306 and 308 such that any imaginary lines made from marks 302 to 306 and from marks 304 to 308 would essentially go right over the impression from the crosshairs.

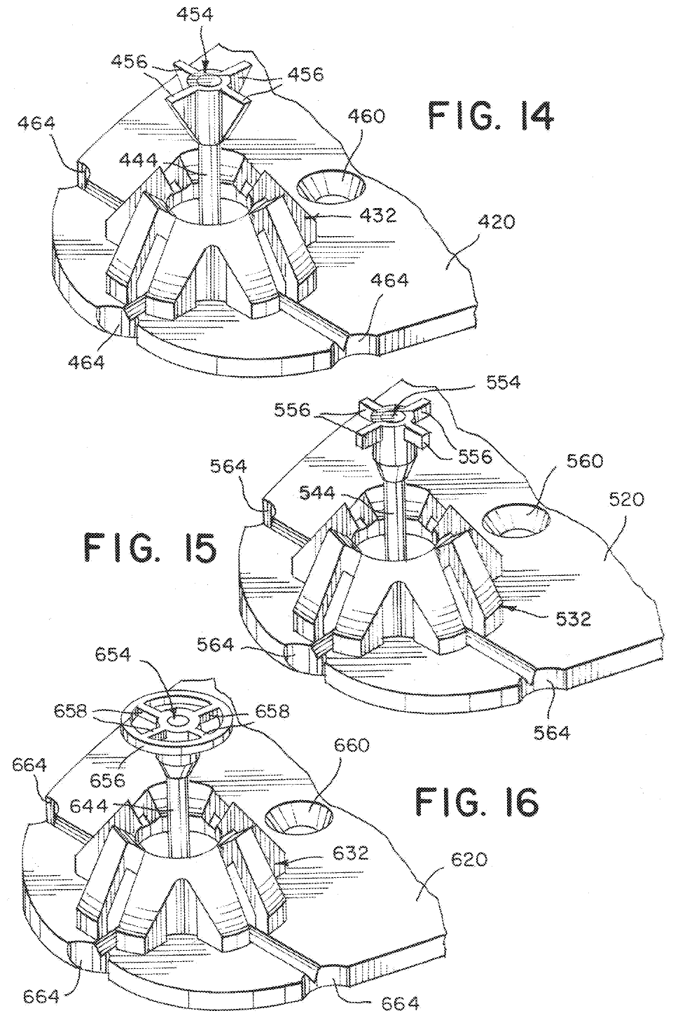

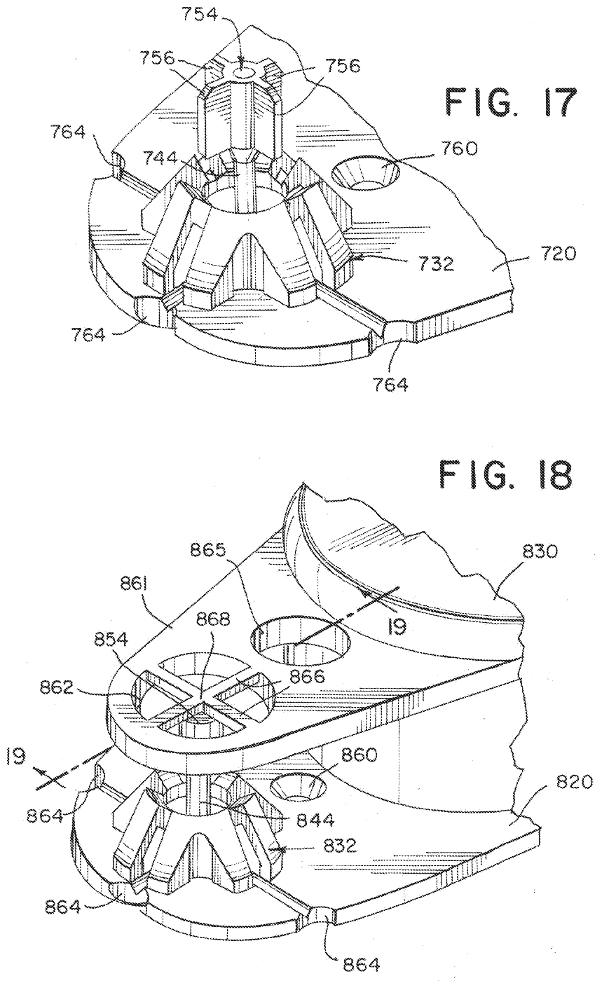

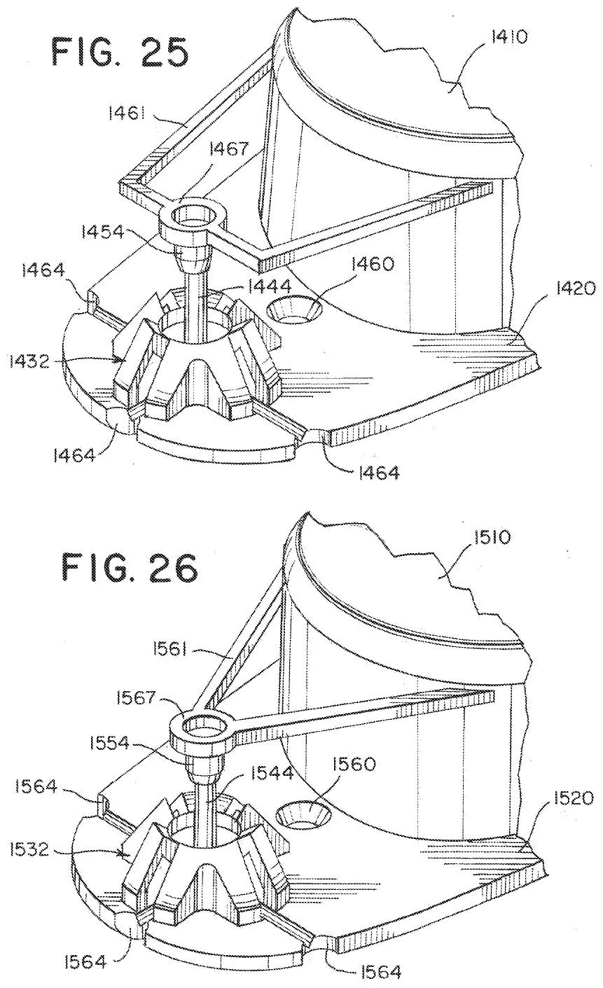

Referring now to FIGS. 14-26, alternative embodiments of the cage and pin are illustrated. Each embodiment could be substituted for use in the embodiments of FIGS. 3-7C, 8-10 and 11-12. For each of these embodiments, only the portions that differ from the previous embodiments will be described. In FIG. 14, cage 432 includes a pin 444 and a position indicator 454. Position indicator 454 has four triangular projections 456 to help the user identify when pin 444 is in the vertical position and aligned above the implant injection port 11 of implant 14. FIG. 15 shows a position indicator 554 has four rectangular shaped projections 556 to help the user identify when pin 544 is in the vertical position and therefore aligned above the implant port. FIG. 16 shows a position indicator 654 has a wheel or ring 656 connected to position indicator 654 by four spokes 658 to help the user identify when pin 644 is in the vertical position and therefore aligned above the implant port. FIG. 17 shows a position indicator 754 has four rectangular shaped projections 756 to help the user identify when pin 744 is in the vertical position and therefore aligned above the implant port. Projections 756 are longer in the axial direction of pin 744 than the rectangular shaped projections 556 of FIG. 15 to aid the user in identifying when pin 744 is aligned above the implant port.

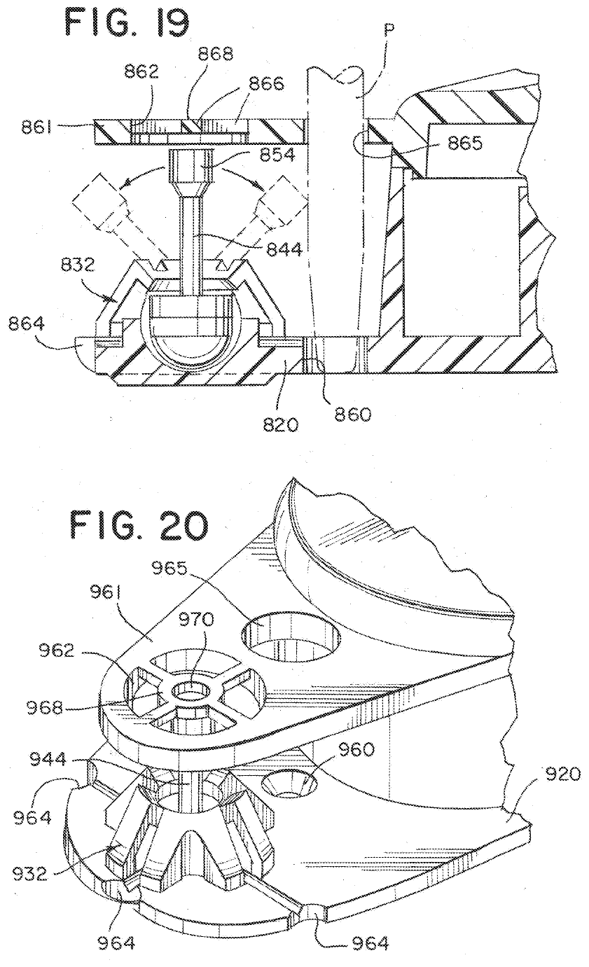

Referring now to FIGS. 18 and 19, an embodiment of cage 832 is illustrated. Pin 844 is disposed under a protective cover 861. Cover 861 may be used to protect pin 844 from damage during shipping, handling and storage. Cover 861 projects outwardly from handle 830 toward pin 844. Cover 861 has a shape that matches the underlying base plate 820. Cover 861 has two through holes or openings 862, 865. Opening 862 has a crosshair shaped arms 866 spanning across opening 862. A center 868 of the crosshair shaped arms 866 is aligned above the center of cage 832. When pin 844 is aligned above the implant port, position indicator 854 is located perpendicular to the plane of plate 820 and is directly under the center 868 of crosshair shaped arms 866 as shown in FIG. 18 and in solid lines in FIG. 19. Opening 865 is aligned above opening 860 in base plate 820. When pin 844 is aligned above the implant port, marks may be made on the skin via through hole 865 and 860 as well as recesses 864 as described above for the embodiment shown and described in FIGS. 3-7C. Similar to what is shown and described in FIG. 13 above, an impression made on the skin 26 of patient 16 from the crosshair shaped projections (not shown in this embodiment, but are preferably as shown and described in FIGS. 3-7C) and marks made on the skin via through hole 860, 865 and recesses 864 may be used by or on the surgeon's behalf to identify where to inject a needle into the skin to add or remove fluid from the implant via the implant port. The crosshair shaped projections can be included in any of the embodiments illustrated in FIGS. 14-26.

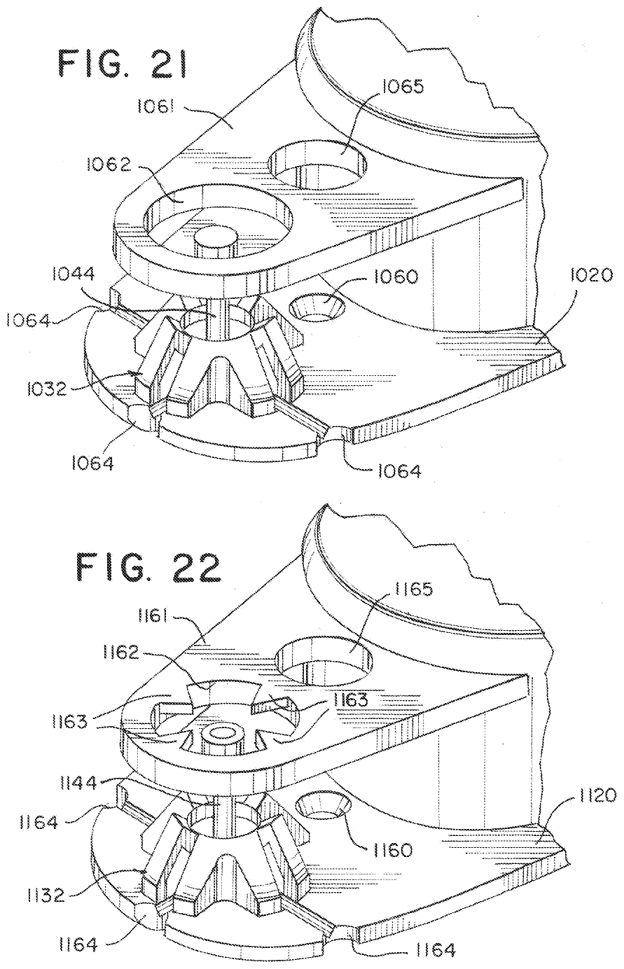

Referring now to FIG. 20, another embodiment of cover 961 is illustrated. Cover 961 differs from cover 861 in that the crosshair shaped arms 966 spanning across opening 962 don't cross at the center but rather have a central circular hub 968 which has a central opening 970 that makes pin 944 readily visible when pin 944 is aligned above the implant port. Thus, the user can readily identify when pin 944 is in the desired location above the implant port by visualizing pin 944 through hole 970 when viewed from above.

Referring now to FIG. 21, another embodiment of cover 1061 is illustrated. Cover 1061 differs from cover 861 and 961 in that the crosshair shaped arms spanning across opening 1062 are eliminated and cover 1061 has a relatively larger opening 1062. Opening 1062 permits pin 1044 to be visible to a user at the center of opening 1062 when pin 1044 is aligned above the implant port. Thus, the user can readily identify when pin 1044 is in the desired location above the implant port by visualizing pin 1044 through hole 1062 when viewed from above.

Referring now to FIG. 22, another embodiment of a cover 1161 is illustrated. Cover 1161 differs from cover 1061 in that opening 1062 has four triangular shaped radially inwardly projecting projections 1163 at approximately 90 degrees symmetrically spaced apart from each other. Opening 1162 has a four-leaf clover shape as shown in FIG. 22. Opening 1162 permits pin 1144 to be visible to a user at the center of opening 1162 when pin 1144 is aligned above the implant port. Thus, the user can readily identify when pin 1144 is in the desired location above the implant port by visualizing pin 1144 through hole 1162 when viewed from above.

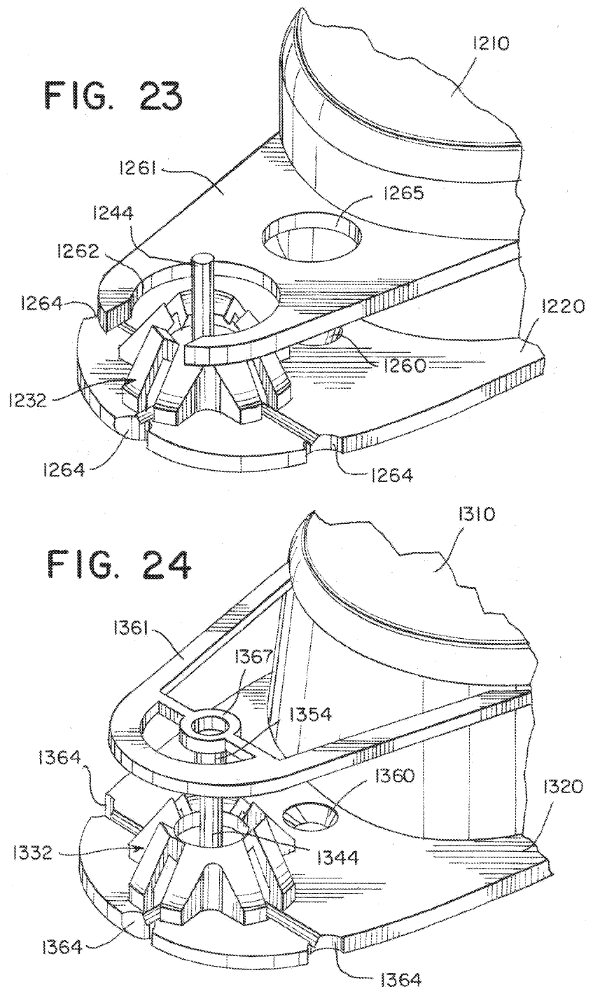

Referring now to FIG. 23, an embodiment of a cover 1261 is illustrated. Cover 1261 is about the same height as a pin 1244. An opening 1262 in cover 1261 is partially open at the end of opposite of handle 1210. Alternatively, opening 1262 could be fully closed like the embodiment of FIG. 21 above.

Referring now to FIGS. 24-26, additional alternative embodiments of the cover are illustrated. In these three embodiments, cover 1361, 1461 and 1561 have minimalistic alignment features to make viewing the pin 1344, 1444 and 1544 easier for the user. In each embodiment the cover 1361, 1461, 1561 extends from handle 1310, 1410, 1510 and has a ring 1367, 1467, 1567 disposed just above position indicator 1354, 1454, 1554. When pin 1344, 1444, 1544 is aligned above the implant port, the user can readily identify when the pin is in the desired location above the implant port by visualizing pin 1344, 1444, 1544 through ring 1367, 1467, 1567 when viewed from above.

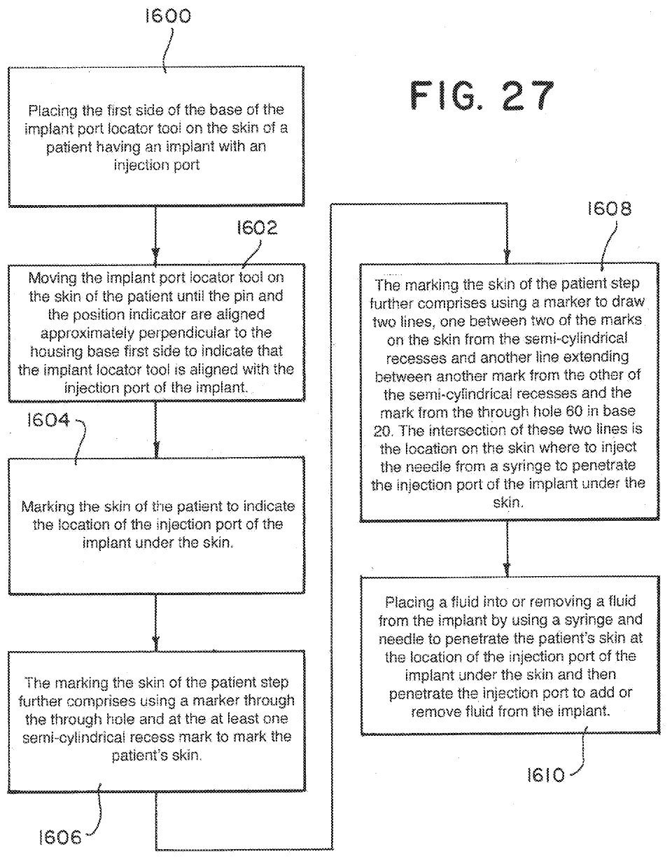

Referring now to FIG. 27, a flow chart illustrating a method for locating an injection port of an implant within a patient with an implant port locator tool is disclosed. The implant port locator tool comprises the following steps:

Placing the first side of the base of the implant port locator tool on the skin of a patient having an implant with an injection port. Step 1600 is placing the first side of the base of the implant port locator tool on the skin of a patient having an implant with an injection port. In step 1602, moving the implant port locator tool on the skin of the patient until the pin and the position indicator are aligned approximately perpendicular to the housing base first side to indicate that the implant locator tool is aligned with the injection port of the implant. In step 1604, marking the skin of the patient to indicate the location of the injection port of the implant under the skin.

Additional steps could include step 1606 which is: the base having a through hole disposed between the handle and the cage, an outer perimeter of the base has at least one semicylindrical recess, wherein the marking the skin of the patient step further comprises using a marker through the through hole and at the at least one semicylindrical recess to mark the patient's skin.

A further additional step 1608 may include wherein the marking the skin of the patient step further comprises using a marker drawing two lines, one between two of the marks on the skin from the semicylindrical recesses and another line extending between another mark from the other of the semicylindrical recesses and the mark from the through hole 60 in base 20. The intersection of these two lines is the location on the skin where to inject the needle from a syringe to penetrate the injection port of the implant under the skin.

A further step 1610 can include placing a fluid into or removing a fluid from the implant by using a syringe and needle to penetrate the patient's skin at the location of the injection port of the implant under the skin and then penetrate the injection port to add or remove fluid from the implant.

The disclosed technology described herein can be further understood according to the following aspects:

Aspect 1. An implant port locator tool for locating an injection port of an implant within a patient, the implant locator tool comprising:
a housing having a base having a first side and a second side, the first side of the base being configured to be placed on the skin of the patient, the second side of the base having a recess;
a handle connected to the second side of the base;
a cage having an outer perimeter connected to the second side of the base, the cage having a recess and a central opening in the recess; wherein the recess in the base and the recess in the cage mate to form a receptacle with the central opening being disposed at an apex of the receptacle, spaced a predetermined distance from the base;
a pin having a first end and a second end; the first end of the pin disposed within the receptacle, the second end of the pin disposed exterior to the recess via the central opening in the cage; and
a magnet connected to the first end of the pin; wherein the magnet and the second end of the pin are captured in the receptacle.

Aspect 2. The implant locator tool according to aspect 1, wherein the recess in the base has a hemispherical shape.

Aspect 3. The implant locator tool according to any of the previous aspects, wherein the recess in the cage has a hemispherical shape.

Aspect 4. The implant locator tool according to aspect 3, wherein the receptacle has a spherical shape.

Aspect 5. The implant locator tool according to aspect 3, wherein the magnet and the first end of the pin are captured in the receptacle in a ball and socket connection.

Aspect 6. The implant locator tool according to any of the previous aspects, wherein the magnet is a spherical magnet.

Aspect 7. The implant locator tool according to any of the previous aspects 1-4, wherein the magnet is an annular ring magnet.

Aspect 8. The implant locator tool according to aspect 7, wherein the magnet is press fit onto the first end of the pin.

Aspect 9. The implant locator tool according to aspect 8, wherein the first end of the pin has an enlarged semispherical head at its distal end.

Aspect 10. The implant locator tool according to aspect 9, wherein the magnet is adjacent to the semispherical head at the first end of the pin.

Aspect 11. The implant locator tool according to any of the previous aspects, wherein the second end of the pin has a position indicator connected to the distal second end of the pin, the pin and the position indicator configured to be aligned approximately perpendicular to the first side of housing base when the implant locator tool is aligned with the injection port of the implant.

Aspect 12. The implant locator tool according to aspect 11, wherein the first side of the base having a crosshair shaped projection projecting away from the first side of the base by a predetermined distance.

Aspect 13. The implant locator tool according to aspect 12, wherein a center of the crosshair shaped projection is aligned with an axis of the pin when the implant locator tool is aligned with the injection port of the implant.

Aspect 14. The implant locator tool according to any of the previous aspects, wherein the base has a through hole disposed between the handle and the cage.

Aspect 15. The implant locator tool according to aspect 14, wherein an outer perimeter of the base has at least one semicylindrical recess.

Aspect 16. The implant locator tool according to aspect 15, wherein the second end of the pin has a position indicator connected to the distal second end of the pin, the position indicator configured to be aligned approximately perpendicular to the housing base first side when the implant locator tool is aligned with the injection port of the implant, the first side of the base having a crosshair shaped projection projecting away from the first side of the base by a predetermined distance, a center of the crosshair shaped projection is aligned with an axis of the pin when the implant locator tool is aligned with the injection port of the implant;

wherein an imaginary line extending from one of the crosshair projections intersects the through hole in the base, wherein an imaginary line extending from one of the crosshair projections intersects the at least one semicylindrical recess in the outer perimeter in the base.

Aspect 17. An implant port locator tool for locating an injection port of an implant within a patient, the implant locator tool comprising:
a first plate having a first side and a second side, the first side of the first plate being configured to be placed on the skin of the patient, the second side of the first plate having a recess;
a second plate having a first side and a second side, the first side of the second plate connected to the second side of the first plate, the first side of the second plate having a recess and a central opening in the recess opening to the second side of the second plate; wherein the recess in the first plate and the recess in the second plate mate to form a receptacle;
a pin having a first end and a second end; the first end of the pin disposed within the receptacle, the second end of the pin disposed exterior to the recess via the central opening in the second plate; and
a magnet connected to the first end of the pin; wherein the magnet and the first end of the pin are captured in the receptacle.

Aspect 18. The implant locator tool according to aspect 17, wherein the recess in the first plate has a hemispherical shape.

Aspect 19. The implant locator tool according to aspect 18, wherein the recess in the second plate has a hemispherical shape.

Aspect 20. The implant locator tool according to aspect 19, wherein the receptacle has a spherical shape.

Aspect 21. The implant locator tool according to aspect 19, wherein the magnet and the first end of the pin are captured in the receptacle in a ball and socket connection.

Aspect 22. The implant locator tool according to any of the previous aspects 17-21, wherein the magnet is an annular ring magnet.

Aspect 23. The implant locator tool according to any of the previous aspects 17-22, wherein the magnet is press fit onto the first end of the pin.

Aspect 24. The implant locator tool according to aspect 23, wherein the first end of the pin has an enlarged semispherical head at its distal end.

Aspect 25. The implant locator tool according to aspect 24, wherein the magnet is adjacent to the semispherical head at the first end of the pin.

Aspect 26. The implant locator tool according to aspect 22, wherein the second end of the pin has a position indicator connected to the distal second end of the pin, the pin and position indicator configured to be aligned approximately perpendicular to the first side of the first plate when the implant locator tool is aligned with the injection port of the implant.

Aspect 27. The implant locator tool according to aspect 26, wherein the first side of the first plate having a crosshair shaped projection projecting away from the first side of the first plate by a predetermined distance.

Aspect 28. The implant locator tool according to aspect 27, wherein a center of the crosshair shaped projection is aligned with an axis of the pin when the implant locator tool is aligned with the injection port of the implant.

Aspect 29. The implant locator tool according to any of the previous aspects 17-28, wherein the first plate and the second plate has four through holes spaced symmetrically about the receptacle.

Aspect 30. The implant locator tool according to aspect 28, wherein the first plate 120 and the second plate has four through holes spaced symmetrically about the receptacle, wherein an imaginary line drawn through the centers of two of the through holes intersects with the center of the crosshair shaped projection.

Aspect 31. The implant locator tool according to aspect 30, wherein an imaginary line drawn through the centers of the other two of the through holes intersects with the center of the crosshair shaped projection.

Aspect 32. The implant locator tool according to any of the previous aspects 17-30, further comprising a handle connected to the first plate.

Aspect 33. The implant locator tool according to any of the previous aspects, wherein the magnet disposed in the injection port of the implant is disc shaped and has a size of less than or equal to about 0.375 inches in diameter and a height of less than or equal to about 0.1875 inches.

Aspect 34. A method of locating an injection port of an implant within a patient with an implant port locator tool, the implant port locator tool comprising:
a housing having a base having a first side and a second side, the first side of the base being configured to be placed on the skin of the patient, the second side of the base having a recess;
a handle connected to the second side of the base;
a cage having an outer perimeter connected to the second side of the base, the cage having a recess and a central opening in the recess; wherein the recess in the base and the recess in the cage mate to form a receptacle with the central opening being disposed at an apex of the receptacle, spaced a predetermined distance from the base;
a pin having a first end and a second end; the first end of the pin disposed within the receptacle, the second end of the pin disposed exterior to the recess via the central opening in the cage; and
a magnet connected to the first end of the pin; wherein the magnet and the second end of the pin are captured in the receptacle;
wherein the method comprising the steps of:
placing the first side of the base of the implant port locator tool on the skin of a patient having an implant with an injection port;
moving the implant port locator tool on the skin of the patient until the pin and the position indicator are aligned approximately perpendicular to the housing base first side to indicate that the implant locator tool is aligned with the injection port of the implant;
marking the skin of the patient to indicate the location of the injection port of the implant under the skin.

Aspect 35. The method of locating an injection port of an implant according to aspect 34, wherein the implant locator tool further comprises the base having a through hole disposed between the handle and the cage, an outer perimeter of the base has at least one semicylindrical recess, wherein the marking the skin of the patient step further comprises:
using a marker through the through hole and the at least one semicylindrical recess to mark the patient's skin.

The descriptions contained herein are examples of embodiments of the invention and are not intended in any way to limit the scope of the invention. As described herein, the invention contemplates many variations and modifications of a port locator tool, including modifications and variations apparent to those having skill in the pertinent art according to the teachings of this disclosure are intended to be within the scope of the claims which follow.

What is claimed is:

1. An implant port locator tool for locating a magnet disposed in an injection port of an implant within a patient, the implant locator tool comprising:
a housing having a base having a first side and a second side, the first side of the base being configured to be placed on the skin of the patient, the second side of the base having a recess;
a handle connected to the second side of the base;
a cage having an outer perimeter connected to the second side of the base, the cage having a recess and a central opening in the recess; wherein the recess in the base and the recess in the cage mate to form a receptacle with the central opening being disposed at an apex of the receptacle, spaced a predetermined distance from the base;
a pin having a first end and a second end; the first end of the pin disposed within the receptacle, the second end of the pin disposed exterior to the recess via the central opening in the cage; and
a magnet connected to the first end of the pin; wherein the magnet and the second end of the pin are captured in the receptacle.

2. The implant locator tool according to claim 1, wherein the recess in the base has a hemispherical shape.

3. The implant locator tool according to claim 2, wherein the recess in the cage has a hemispherical shape.

4. The implant locator tool according to claim 3, wherein the receptacle has a spherical shape.

5. The implant locator tool according to claim 3, wherein the magnet and the first end of the pin are captured in the receptacle in a ball and socket connection.

6. The implant locator tool according to claim 1, wherein the magnet is an annular ring magnet.

7. The implant locator tool according to claim 1, wherein the magnet is a spherical magnet.

8. The implant locator tool according to claim 6, wherein the magnet is press fit onto the first end of the pin.

9. The implant locator tool according to claim 8, wherein the first end of the pin has an enlarged semispherical head at its distal end.

10. The implant locator tool according to claim 9, wherein the magnet is adjacent to the semispherical head at the first end of the pin.

11. The implant locator tool according to claim 6, wherein the second end of the pin has a position indicator connected to the distal second end of the pin, the pin and the position indicator configured to be aligned approximately perpendicular to the first side of housing base when the implant locator tool is aligned with the injection port of the implant.

12. The implant locator tool according to claim 11, wherein the first side of the base having a crosshair shaped projection projecting away from the first side of the base by a predetermined distance.

13. The implant locator tool according to claim 12, wherein a center of the crosshair shaped projection is aligned with an axis of the pin when the implant locator tool is aligned with the injection port of the implant.

14. The implant locator tool according to claim 1, wherein the base has a through hole disposed between the handle and the cage.

15. The implant locator tool according to claim 14, wherein an outer perimeter of the base has at least one semicylindrical recess.

16. The implant locator tool according to claim 15, wherein the second end of the pin has a position indicator connected to the distal second end of the pin, the position indicator configured to be aligned approximately perpendicular to the housing base first side when the implant locator tool is aligned with the injection port of the implant, the first side of the base having a crosshair shaped projection projecting away from the first side of the base by a predetermined distance, a center of the crosshair shaped projection is aligned with an axis of the pin when the implant locator tool is aligned with the injection port of the implant;
wherein an imaginary line extending from one of the crosshair projections intersects the through hole in the base, wherein an imaginary line extending from one of the crosshair projections intersects the at least one semicylindrical recess in the outer perimeter in the base.

17. An implant port locator tool for locating an injection port of an implant within a patient, the implant locator tool comprising:
a first plate having a first side and a second side, the first side of the first plate being configured to be placed on the skin of the patient, the second side of the first plate having a recess;
a second plate having a first side and a second side, the first side of the second plate connected to the second side of the first plate, the first side of the second plate having a recess and a central opening in the recess opening to the second side of the second plate; wherein the recess in the first plate and the recess in the second plate mate to form a receptacle;
a pin having a first end and a second end; the first end of the pin disposed within the receptacle, the second end of the pin disposed exterior to the recess via the central opening in the second plate; and
a magnet connected to the first end of the pin; wherein the magnet and the first end of the pin are captured in the receptacle.

18. The implant locator tool according to claim 17, wherein the recess in the first plate has a hemispherical shape.

19. The implant locator tool according to claim 18, wherein the recess in the second plate has a hemispherical shape.

20. The implant locator tool according to claim 19, wherein the receptacle has a spherical shape.
